(19) United States
(12) Patent Application Publication
(10) Pub. No.: US 2002/0037933 A1
(43) Pub. Date: Mar. 28, 2002

(54) Management of septic shock

(76) Inventors: Samar Basu, Uppsala (SE); Arne Eek, Sodertalje (SE); Mats Eriksson, Uppsala (SE)

Correspondence Address: PILLSBURY WINTHROP LLP, 1600 TYSONS BOULEVARD, MCLEAN, VA, 22102, US

(21) Appl. No.: 09/849,591

(22) Filed: May 7, 2001

(30) Foreign Application Priority Data
May 19, 2000 (SE) 0001865-5

(51) Int. Cl. A61K 31/05

(52) U.S. Cl. 514/731

(57) ABSTRACT

The invention relates to a method of managing septic shock and counteracting endotoxin induced deterioration of arterial oxygen tension which comprises administration of an effective amount of a sterile pharmaceutical composition for parenteral administration, which composition comprises the compound 2,6-diisopropylphenol (propofol) in association with a sterile pharmaceutically-acceptable diluent or carrier, and the use of such a sterile pharmaceutical composition for use as a medicament for managing septic shock, and the use of such a sterile pharmaceutical composition for the manufacture of a medicament for the management of septic shock and for counteracting endotoxin induced deterioration of arterial oxygen tension.

The present invention relates to septic shock, a method of managing septic shock and counteracting endotoxin induced deterioration of arterial oxygen tension, and the use of certain sterile pharmaceutical compositions for the manufacture of a medicament for the management of septic shock and for counteracting endotoxin induced deterioration of arterial oxygen tension.

In the US alone, approximately 500,000 people per year suffer from sepsis, of which it is estimated 175,000 will die (Stone R., Science, 1994, 264, 365-7). Despite advances in antimicrobial therapy and medical support, septic shock remains a leading cause of death in intensive care units (ICUs), and its incidence is increasing. High mortality rates are reported (such as 95%), which can be as high as 60% even with prompt diagnosis and treatment. For a review of septic shock, its pathogenesis and current treatment see, for example, Wiessner W. H., Casey L. C. & Zbilut J. P., Heart Lung, 1995, 24(5), 380-92; Meier-Hellmann A. Et al, Clin.Chem.Lab.Med., 1999, 37(3), 333-9; Dellinger R. P., Infect Dis.Clin.North Am., 1999, 13(2), 495-509; Hazinski M.F., Crit.Care Nurs.Clin.North Am., 1994, 6(2), 309-19; Zanetti G. Et al, Schweiz Med.Wochenschr., 1997, 127(12), 489-99 and Oh H. M., Ann.Acad.Med. Singapore, 1998, 27(5), 738-43.

In outline, Gram-negative septic shock arises when endotoxin is released from Gram negative bacteria following a serious infection. Difficulties in keeping up the arterial oxygen tension is one of the characteristics in septic shock, and the condition deteriorates frequently, leading to possible development of adult respiratory distress syndrome (ARDS). This serious condition includes difficulty in keeping the patient oxygenated, as the lungs are heavily affected and oedematous. The endotoxin initiates a highly complex cascade of biochemical processes involving cytokine complement and/or arachidonic acid, the latter leading to several pathophysiological events, including free radical mediated oxidative injury and/or COX mediated inflammation. Two parameters believed to be implicated in septic shock are the prostaglandins and isoprostanes (a subgroup of prostaglandins, mainly PGF2α and 8-iso-PGF2α) which are mainly metabolised in the lungs (a target organ in ARDS). The endotoxin also induces dilation of blood arteries and arterioles, leading to a reduction in circulating blood volume to vital organs such as the brain. Thus, in septic shock caused by Gram-negative bacteria endotoxin causes a deterioration of arterial oxygen tension. However, septic shock may also be caused by Gram-positive bacteria, fungi etc. In these cases endotoxin is not nvolved.

The interactions and cascades of events in septic shock are complex and there is currently no effective treatment available to intervene in these events once sepsis and septic shock have occurred. Current therapy for septic shock comprises the administration of antibiotics and fluids, and treatment to maintain blood pressure (for example, by using ionotropes). There is often additional treatment for other attendant symptoms.

Emerging adjunctive therapy for septic shock can be divided into those treatments directed against bacterial components, those directed against host-derived inflammatory-mediators and those designed to limit tissue damage. All trials of new adjunctive therapies for sepsis and septic shock conducted to date have failed to show efficacy. Therapies tried against endotoxin, including tumour necrosis factor, interleukin-1 and platelet activating factor, have not reduced mortality in septic shock. Possible future effective therapies may use a combination of agents depending upon the nature of the infection and the type of patient. However, there remains a strong unmet need for an effective therapy for the management of septic shock.

We have now surprisingly found that 2,6-diisopropylphenol (propofol) is effective in counteracting endotoxin induced deterioration of arterial oxygen tension in pigs, and that propofol and pharmaceutical compositions containing propofol may thus be of value as a therapy for the management of septic shock.

Propofol is an intravenous sedative agent and can be used for the induction and maintaince of general anaesthesia and for sedation, for example in Intensive Care Units. Propofol is a highly successful anaesthetic and is marketed under the trademark ‘Diprivan’ for use in treating humans and under the trademark ‘Rapinovet’ for veterinary use.

Once the anaesthetic properties of propofol were identified, UK patent application Ser. No. 13739/74 was filed and this was granted as UK Patent 1,472,793. Corresponding patents have been granted in the USA (U.S. Pat. No. 4,056,635, U.S. Pat. No. 4,452,817 and U.S. Pat. No. 4,798,846) and many other territories.

The finding that adventitious extrinsic microbial contamination resulting from non-asceptic handling of the original formulation of ‘Diprivan’ could lead to post-operative infection, initiated development of a modified formulation with a suitable additive present (the additive being capable of retarding the growth of common micro-organisms to not greater than 1 log increase (ie, 10 fold) in 24 hours following extrinsic contamination equivalent to ‘touch contamination’). The development of a modified, edetate-containing formulation of propofol led to the filing and grant, inter alia, of UK Patent No. 2,298,789. Corresponding patents have been granted, for example, in the USA (U.S. Pat. No. 5,714,520; U.S. Pat. No. 5,731,355; U.S. Pat. No. 5,731,356; U.S. Pat. No. 5,908,869) and in other territories. The marketed modified formulation of ‘Diprivan’ contains 0.005% disodium edetate.

As stated above, and illustrated in the accompanying non-limiting Experiments and Results section, we have surprisingly found that pharmaceutical compositions containing propofol are effective in counteracting endotoxin induced deterioration of arterial oxygen tension in pigs, and that propofol and pharmaceutical compositions containing propofol are of value in the management of septic shock. Studies comparing the use of propofol with other sedatives (eg. Midazolam) in ICUs have focused on objectives such as cost, weaning from ventilation and nutrition. No study has demonstrated that propofol counteracts endotoxin induced deterioration of arterial oxygen tension, and thus may be of value for the management of septic shock or Gram-negative septic shock.

By ‘management of septic shock’ we mean treatment, including prophylactic treatment or prevention, of some or all of the effects of septic shock, in particular Gram-negative septic shock. The term also includes management of severe sepsis (without or without shock) when a deterioration of arterial oxygen tension is present in such generalised systemic sepsis (sepsis syndrome). Thus, the term includes, a reduction in the effects of sepsis syndrome and/or septic shock, and assistance in recovery from sepsis syndrome and/or septic shock. Some of the effects of sepsis syndrome and/or septic shock which may be managed include blood pressure (which may be raised to more healthy levels), body temperature (which may be normalised) and left-sided pressure as measured, for example, by a Swan-Ganz catheter (which may be normalised). Effective management should also demonstrate improvements/normalisation in arterial oxygen tension (i.e. return towards a baseline level for a healthy subject) and normalisation (i.e. return towards a baseline level for a healthy subject) of prostaglandin 8-iso-PGF2α levels. By way of illustration, normal levels of PaO2 in healthy pigs breathing air (ca. 21% oxygen) are in the range 9.7-12.6 kPa-Ref. Hannon J Bossone C Wade C: Normal physiologic values for conscious pigs used in biomedical research. Lab Animal Sci, 40:293-298, 1990. Normalisation in this context means returning towards such baseline values. The animals in this work were at baseline anaesthetised and mechanically ventilated with 30% oxygen so the baseline values are not synonymous to those found in conscious pigs. However, the values are in the same range or somewhat higher in the experimental pigs. An analgous discussion may be applied to 8-iso-PGF2α levels and to human (versus animal) subjects. An analgous discussion may also be applied to the term “counteracting endotoxin induced deterioration of arterial oxygen tension” (i.e. return towards a baseline arterial oxygen tension level for a healthy subject).

Without wishing to be constrained by theory, we believe the effects described herein to result from a reduction in expressed oxidative injury, with propofol believed to have an effect on both free radical and COX-mediated injury. The effect in counteracting endotoxin induced deterioration of arterial oxygen tension is thought to arise from the counteracting of F2-isoprostane formation by the scavenging of free radicals. This is thought to effect a reduction in lipid peroxidation, mainly F2-isoprostane formation, and may also make propofol and pharmaceutical compositions containing propofol useful for the management and treatment of reperfusion injury after, for example, cardiac arrest and/or vascular surgery. Propofol has the potential to keep up oxygen tension in arterial blood, and possibly also in tissues, in conditions which are secondary to free radical mediated injury. Such conditions may include not only septic shock, but also be present in bowel surgery patients, irradiation injury, burn wounds, vascular injury and cardiac arrest. Propofol may thus have benefit in a wide range of injuries as well as being of benefit in the mangement (including prophylactic use) of septic shock. Administration of propofol may be systemic or topical depending on the setting.

Given the particular complexity of interactions involved in septic shock, the effectiveness of propofol in counteracting the complex cascade of events is genuinely surprising. Indeed, given that hypotension is a possible adverse event (depending on dose and use of premedications and other agents) when using pharmaceutical compositions containing propofol, it may be considered yet more surprising that the endotoxin induced deterioration of arterial oxygen tension studied here was improved rather than worsened (reduction in blood pressure is one of the characteristics of endotoxaemia and septic shock).

The results reported here show, for the first time, that 2,6-diisopropylphenol (propofol) in both original formulation and modified formulation Diprivan (containing disodium edetate) has a rapid effect in counteracting (i.e. returning towards normal baseline levels) endotoxin induced deterioration of arterial oxygen tension in endotoxaemic pigs (endotoxin infusion is frequently used to mimic Gram-negative septic shock). Furthermore, plasma concentration of 8-iso-PGF2α did not increase when using original formulation Diprivan. No deaths from ypotension and/or release of gut bacteria into the bloodstream were observed in the already anaethesised animals studied in this work.

The observed effects on arterial oxygen tension were similar whether the propofol formulation contained disodium edetate or not.

An increase in γ-tocopherol levels was observed in this work when using original formulation Diprivan (no plasma analysis was performed when modified Diprivan), but not until the later phase of the experiments. The patterns of both α-tocopherol and γ-tocopherol were completely different from the patterns of 8-iso-PGF2α and 15-k-dh-PGF2α respectively, indicating that the beneficial effect of propofol is not secondary to γ-tocopherol. Accordingly, the observed effect is not believed to arise from the presence of (disodium) edetate and/or γ-tocopherol. This is in contrast to the beneficial effects of propofol on arterial oxygen tension and the plasma concentration of 8-iso-PGF2α, which were siginificant after a short time of endotoxaemia.

Accordingly, the present invention provides a method of managing septic shock which comprises administration of an effective amount of a pharmaceutical composition as described and claimed in United Kingdom Patents 1,472,793 or 2,298,789.

In particular the present invention provides a method of managing septic shock which comprises administration of an effective amount of a sterile pharmaceutical composition which composition comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal.

The invention further provides a method of managing septic shock which comprises administration of an effective amount of a sterile pharmaceutical composition for parenteral administration which composition comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination).

The invention also provides a pharmaceutical composition as described and claimed in United Kingdom Patents 1,472,793 or 2,298,789 for use as a medicament for managing septic shock.

The invention further provides a sterile pharmaceutical composition which comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal, for use as a medicament for managing septic shock.

The invention further provides a sterile pharmaceutical composition for parenteral administration which comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination), for use as a medicament for managing septic shock.

The present invention also provides the use of a pharmaceutical composition as described and claimed in United Kingdom Patents 1,472,793 or 2,298,789 for the manufacture of a medicament for managing septic shock.

In particular the present invention provides the use of a sterile pharmaceutical composition which comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal for the manufacture of a medicament for managing septic shock.

The invention further provides the use of a sterile pharmaceutical composition for parenteral administration which comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination) for the manufacture of a medicament for managing septic shock.

The present invention provides a method of counteracting endotoxin induced deterioration of arterial oxygen tension which comprises administration of an effective amount of a pharmaceutical composition as described and claimed in United Kingdom Patents 1,472,793 or 2,298,789.

In particular the present invention provides a method of counteracting endotoxin induced deterioration of arterial oxygen tension which comprises administration of an effective amount of a sterile pharmaceutical composition which composition comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal.

The invention further provides a method of counteracting endotoxin induced deterioration of arterial oxygen tension which comprises administration of an effective amount of a sterile pharmaceutical composition for parenteral administration which composition comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination).

The invention further provides a use and method as described herein, achieved substantially as described in the Experiments and Results section herein.

In a particular embodiment the use and method of the invention are used for prophylactic management of septic shock.

The methods and uses of the present invention relate to use in warm-blooded animals, in particular such as man, requiring the counteracting of endotoxin induced deterioration of arterial oxygen tension, and/or the management of septic shock. In particular, the methods and uses of the present invention relate to the management of Gram-negative septic shock.

The present invention thus provides, for example, the following:

(a) A sterile pharmaceutical composition which comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal, for use as a medicament for managing septic shock.

(b) A sterile pharmaceutical composition according to (a), in which the sterile pharmaceutical composition comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination), for use as a medicament for managing septic shock.

(c) The use of the compound 2,6-diisopropylphenol (propofol) for the manufacture of a medicament for managing septic shock.

(d) The use of the compound 2,6-diisopropylphenol (propofol) for the manufacture of a medicament for counteracting endotoxin induced deterioration of arterial oxygen tension.

(e) The use of a sterile pharmaceutical composition which comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal, for the manufacture of a medicament for managing septic shock.

(f) The use of a sterile pharmaceutical composition which comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination) for the manufacture of a medicament for managing septic shock.

(g) The use of a sterile pharmaceutical composition according to (e) and (f), for the manufacture of a medicament for counteracting endotoxin induced deterioration of arterial oxygen tension.

(h) The use according to any one of (e) to (g), in which the sterile pharmaceutical composition is in the form of an oil-in-water emulsion which comprises:

(1) 1% by weight of propofol,

(2) 10% by weight of soy bean oil,

(3) 1.2% by weight of egg phosphatide,

(4) 2.25% by weight of glycerol,

(5) sodium hydroxide,

(6) water.

(i) The use according to any one of (e) to (g), in which the sterile pharmaceutical composition is in the form of an oil-in-water emulsion which comprises:

(1) 2% by weight of propofol,

(2) 10% by weight of soy bean oil,

(3) 1.2% by weight of egg phosphatide,

(4) 2.25% by weight of glycerol,

(5) sodium hydroxide,

(6) water.

(j) The use according to (h) or (i), in which the sterile pharmaceutical composition additionally contains 0.005% by weight of disodium edetate.

(k) A method of managing septic shock which comprises administration of an effective amount of a sterile pharmaceutical composition which composition comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal.

(l) A method according to (k), in which the sterile pharmaceutical composition comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination).

(m) A method of counteracting endotoxin induced deterioration of arterial oxygen tension which comprises administration of an effective amount of a sterile pharmaceutical composition, which composition comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal.

(n) A method according to (m), in which the sterile pharmaceutical composition comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination).

By ‘pharmaceutical compositions containing propofol’ we include those pharmaceutical compositions described and claimed in United Kingdom Patents 1,472,793 and 2,298,789 (the contents of both of which are hereby incorporated by reference), and corresponding applications/patents in other territories. Provided that propofol is present in a composition suitable for administration, other additives may also be present (see later under “Combination with other therapeutic agents”). Also included in the present invention are compositions comprising pro-drugs of propofol, which may be metabolised to provide propofol per se in-vivo.

By an ‘oil-in-water emulsion’ we mean a distinct two-phase system that is in equilibrium and in effect, as a whole, is kinetically stable and thermodynamically unstable.

By the term ‘edetate’ we include metal ion chelating/sequestering agents, such as polyaminocarboxylate chelators, such as ‘edetate’ (ethylenediaminetetraacetic acid-EDTA), diethylenetriaminepentaacetic acid (DTPA) and EGTA, and derivatives thereof. For example, the disodium derivative of edetate is known as disodium edetate. In general, suitable metal ion chelating agents are those salts having lower affinity for the free acid form than calcium, and in particular those derivatives descibed in UK Patent No. 2,298,789. A particular, preferred metal ion chelating agent is disodium edetate.

In propofol compositions containing edetate, typically the metal ion chelating agent will be present in the compositions in a molar concentration (with respect to the metal ion chelating agent free acid) in the range 3×10-5 to 9×10-4. Preferably the metal ion chelating agent free acid is present in the range 3×10-5 to 7.5×10-4, for example in the range 5×10-5 to 5×10-4 and more preferably in the range 1.5×10-4 to 1.5×10-4, most preferably about 1.5×10-4. In particular, the metal ion chelating agent free acid is present in the range from about 0.0005% to 0.1% (the precise concentration depending upon the properties of the metal ion chelating agent selected, provided significant growth of microorganisms for at least 24 hours is prevented in the event of adventitious, extrinsic contamination).

A propofol composition suitable for use according to the present invention typically comprises from 0.1 to 5%, by weight, of propofol. Preferably the composition comprises from 1 to 2% by weight of propofol and, in particular, about 1% or about 2%. Propofol alone may be emulsified with water by means of a surfactant, but it is preferred that propofol is dissolved in a water-immiscible solvent prior to emulsification. The water-immiscible solvent is suitably present in an amount that is up to 30% by weight of the composition, more suitably 5-25%, preferably 10-20% and in particular about 10%.

A wide range of water-immiscible solvents can be used in the compositions suitable for use in the present invention. Typically the water-immiscible solvent is a vegetable oil, for example soy bean, safflower, cottonseed, corn, sunflower, arachis, castor or olive oil. Preferably the vegetable oil is soy bean oil. Alternatively, the water-immiscible solvent is an ester of a medium or long-chain fatty acid for example a mono-, di-, or triglyceride; or is a chemically modified or manufactured material such as ethyl oleate, isopropyl myristate, isopropyl palmitate, a glycerol ester or polyoxyl hydrogenated castor oil. In a further alternative the water-immiscible solvent may be a marine oil, for example cod liver or another fish-derived oil. Suitable solvents also include fractionated oils for example fractionated coconut oil or modified soy bean oil. Furthermore, the compositions suitable for use in the present invention may comprise a mixture of two or more of the above water-immiscible solvents.

Propofol, either alone or dissolved in a water-immiscible solvent, is emulsified by means of a surfactant. Suitable surfactants include synthetic non-ionic surfactants, for example ethoxylated ethers and esters and polypropylene-polyethylene block co-polymers, and phosphatides for example naturally occuring phosphatides such as egg and soya phosphatides and modified or artificially manipulated phosphatides (for example prepared by physical fractionation and/or chromatography), or mixtures thereof. Preferred surfactants are egg and soya phosphatides.

The compositions suitable for use in the present invention are suitably formulated to be at physiologically neutral pH, typically in the range 6.0-8.5, if necessary by means of alkali such as sodium hydroxide.

The compositions suitable for use in the present invention may be made isotonic with blood by the incorporation of a suitable tonicity modifier for example glycerol.

The compositions suitable for use in the present invention are typically sterile formulations and are prepared according to conventional manufacturing techniques using for example aseptic manufacture or terminal sterilisation by autoclaving. Further details on the preparation of compositions suitable for use in the present invention are included in the Patents referred to herein in the introduction, and are hereby incorporated by reference.

The compositions suitable for use in the present invention are useful as anaesthetics, which includes sedation and induction and maintenance of general anaesthesia, and such properties may be usefully exploited during the management of septic shock according to the present invention. Propofol is a short-acting anaesthetic, suitable for both induction and maintenance of general anaesthesia, for sedation to supplement regional analgesic techniques, for sedation of ventilated patients receiving intensive care and for conscious sedation for surgical and diagnostic procedures in Intensive Care Units. Propofol may be administered by single or repeated intravenous bolus injections or by continuous infusion. It is very rapidly removed from the blood stream and metabolised. Thus the depth of sedation is easily controlled and patient recovery on discontinuing the drug is usually rapid and the patient is often significantly more clear headed as compared to after administration of other anaesthetics.

Dosage levels of propofol for producing general anaesthesia, both induction (for example about 2.0-2.5 mg/kg for an adult human) and maintenance (for example about 4-12 mg/kg/hr), and for producing conscious sedation and/or ICU sedation (for example 0.3-4.5 mg/kg/hr), may be derived from the substantial literature on propofol. For human children, and other animals (such as pigs), higher doses may be required (for example, 5.0-7.5 mg/kg for induction), and up to ca. 24 mg/kg/hr for maintenance. Furthermore the anaesthetist and/or physician would modify the dose to achieve the desired effect in any particular patient, in accordance with normal skill in the art. The dosage levels suitable for treatment or prevention of septic shock are generally within those indicated above (e.g. 0.3-12 mg/Kg/hr for adult humans), but may be optimised to achieve the desired effect in any particular patient, in accordance with normal skill in the art (for example, by determination of the dose at which the arterial oxygen tension, and/or other measure of septic shock recovers towards a normal healthy level). For example, for an adult human, suitable levels (based on those levels used for pigs in the experiments reported herein) are about 2.0-2.5 mg/kg for induction (over about 5 minutes) followed by a continuous infusion at about 3.0-6.0 mg/kg/hr. An anaesthetic dose level is recommended.

In use, the propofol composition may be administered for longer than is used for simple sedation, i.e. a patient suffering from septic shock will be simultaneously sedated and treated for septic shock by the administration of the propofol composition, but will be maintained under sedation until it is considered that effective treatment of the septic shock has been delivered. Artificial ventilation requires sedation, and propofol can be used for this purpose. Simultaneously, propofol can improve arterial oxygen tension by a mechanism that is independent of the artificial ventilation. Thus, the value of using intravenous propofol in patients suffering from septic shock may be two-fold. Namely, the avoidance of an unhealthy reduction in arterial oxygen tension, which can be biochemically characterised as an increase in 8-iso-PGF2α (an index of oxidative injury) & possibly also a more moderate increase in COX-mediated 15-k-dh-PGF2α, and secondly, the sedation of patients requiring artificial ventilation.

Combination with Other Therapeutic Agents

As a further feature of the present invention there are provided pharmaceutical compositions containing propofol suitable for use in the present invention for parenteral administration which comprises, for example, an oil-in-water emulsion, containing a therapeutic or pharmaceutical agent, in which the agent, either alone or dissolved in a water-immiscible solvent, is emulsified with water and propofol, and stabilised by means of a surfactant and which optionally further comprises an amount of edetate sufficient to prevent significant growth of microorganisms for at least 24 hours.

Suitable therapeutic or pharmaceutical agents are those capable of being administered parenterally in an oil-in-water emulsion. Typically such agents (which may be administered separately, sequentially or simultaneously with the propofol composition) are lipophilic compounds and may for example be antifungal agents, anaesthetics, antibacterial agents, anti-cancer agents, anti-emetics, antioxidants, agents acting on the central nervous system such as diazepam, steroids, barbiturates and vitamin preparations. The agents most useful are those which may have additional benefit in the treatment or prevention of septic shock and its symptoms and causes, for example, antibacterial agents, NSAIDs, Vitamin E, fluid therapy and vasoactive amines. Supportive treatment of organ insufficiency may include artificial ventilation and dialysis.

Thus, there is provided the use of a pharmaceutical compositions containing propofol for parenteral administration which comprises, for example, an oil-in-water emulsion, containing a therapeutic or pharmaceutical agent, in which the agent, either alone or dissolved in a water-immiscible solvent, is emulsified with water and propofol, and stabilised by means of a surfactant and which optionally further comprises an amount of edetate sufficient to prevent significant growth of microorganisms for at least 24 hours, for the manufacture of a medicament for the treatment or prevention of septic shock. Also provided is a method of treating or preventing septic shock comprising the use of such compositions. In particular this feature of the present invention relates to such oil-in-water emulsions which typically are administered, to patients in need thereof, over periods of a day or more.

Comments herein relating to typical and preferred propofol compositions for use in the present invention and the preparation thereof apply mutatis mutandis to oil-in-water emulsions containing an additional therapeutic or pharmaceutical agent.

Experiments & Results

Materials and Methods

In brief, ten anesthetized mini-pigs were randomly divided into two equally sized groups and given either iv propofol, or the corresponding volume of a soy bean fat emulsion. Endotoxemia (experimental septic shock) was induced by a continuous infusion of E. coli endotoxin.

In detail, ten healthy pigs (both sexes: 10 to 12 weeks of age; between 19.0 and 26.9 kg in weight) were included in the experiment, with approval (C212/98) of the Animal Ethics committee of Uppsala University. Each pig was given an intramuscular injection of 6 mg.kg-1 of Zoletil forte vet® (Zoletil 100®; Tilétamine-Zolazépam; Boehringer Ingleheim Vetmedica, Ingelheim, Germany) mixed with 2.2 mg.kg-1 of Rompun Vet® (Xylazin; Bayer, Leverkusen, Germany) and 0.04 mg.kg-1 of atropine in order to induce anaesthesia. A continuous intravenous infusion of sodium pentobarbital (Apoteksbolaget, Umeå, Sweden; 8 mg.kg-1.h-1) was given in order to maintain anaesthesia. Morphine (20 mg; Pharmacia, Uppsala, Sweden) was injected iv. When the animals had fallen asleep, a tracheotomy was performed. During the experimental period, 2.5% glucose with sodium chloride (70 mmol. 1-1) was infused at a rate of 18 ml.kg-1.h-1. Surgical procedures, comprising the application of an arterial cannula for measuring arterial blood pressure and sampling (into the common right carotid artery); a 7F Swan-Ganz-catheter equipped with a thermistor for measuring of pulmonary arterial blood pressure (into the pulmonary artery); a central venous line for drug infusion (into the right external jugular vein) and a urinary catheter, were performed. Oxygen (30%) was given in N2O during the insertion of catheters otherwise oxygen (30%) was administered in N2. The experimental procedures have previously been described in detail (Eriksson M. et al; Thromb Haemost, 1998; 80, 1022-1026), and are hereby incorporated by reference.

The animals were randomised into two equally sized groups by the sealed envelope method. The pigs followed two experimental protocols as follows.

First Experiment:

Ten pigs were given a continuous infusion of endotoxin, 5 of which received Propofol (original formulation Diprivan, purchased from a Swedish pharmacy source); and 5 of which received the corresponding volume of a 10% soy bean fat emulsion (Vasolipid®, Braun AG, Melsungen, Germany-Vasolipid® in this experiment is considered equivalent to Intralipid®). The pigs in the latter group served as controls.

Second experiment:

This was identical to the first experiment with the execption that 5 pigs received Propofol (modified formulation Diprivan containing 0.005% disodium edetate, supplied by AstraZeneca UK Limited) and 5 pigs received the corresponding volume of a 10% soy bean fat emulsion (Vasolipid®, Braun AG, Melsungen, Germany mixed with 0.005% disodium edetate served as a control-Vasolipid® in this experiment is considered equivalent to Intralipid®). The pigs in the latter group served as controls.

It has been shown that PGF2α, a cyclooxygenase catalysed oxidation product of arachidonic acid, is released during acute inflammation (Basu, Prost.Leuk & Ess.Fatty Acids, 58, 347-352, 1998) and 8-iso-PGF2α, a free radical catalysed oxidation product of arachidonic acid, is released during oxidative injury (Morrow & Roberts, Biochem.Pharma., Col.51, 1-9, 1996). Measurements of 8-iso-PGF2α and 15-k-dh-PGF2α (a major metabolite of PGF2α) have been shown to be good indicators of oxidative injury and inflammation respectively (Basu, Prost.Leuk & Ess.Fatty Acids, 58, 319-325 and 347-352, 1998). In each experiment the pigs were mechanically ventilated and respiratory and circulatory variables were monitored. The plasma levels of 8-iso-PGF2α (an index of oxidative injury) and 15-k-dh-PGF2α (an index of COX-mediated inflammatory response) were measured, as well as the levels of α-tocopherol and γ-tocopherol.

Propofol was administered as follows: Five minutes before the start of the endotoxin infusion, propofol (at 2.5 mg.kg-1) was given iv over 5 min. followed by a continuous propofol infusion at 10 mg.kg-1.h-1. This dosage is within a clinically relevant range (Mathy-Hartert M. et al; Mediat Inflamm, 1998, 7, 327-333), and the typical concentration of propofol in man is exceeded by the present administration (Gepts E. et al; Anesth Analg, 1987, 66, 1256-1263) by a margin such that species differences in propofol elimination should not be of major importance in our model (Simons P.J. et al; Xenobiotica, 1991, 21, 1243-1256). Endotoxemia was induced in all pigs by a continuous endotoxin infusion (E.coli 0111: B4: Sigma Chemicals, St Louis, Mo., USA) at 4 μg.kg-1 for 30 minutes, followed by 1 μg.kg-1.h-1 for a further 5.5 hours.

Arterial blood samples were collected inmmediately before the loading dose of propofol, 30 min. after the start of the endotoxin infusion and at every full hour of endotoxemia. After centrifugation (3000 r.min-1 for 10 min.) the plasma samples were frozen at minus 70° C. for further analysis.

Respiratory and circulatory parameters were observed and registered throughout the experiment, showing a fast onset of endotoxemia, as evaluated by a doubled mean pulmonary arterial pressure (MPAP; mmHg) after about 30 minutes of endotoxin infusion in all animals and deterioration of these variables. The animals that survived 6 hours of endotoxemia were, still under anaesthesia, sacrificed by an iv overdose of potassium chloride. Physiological data were essentially identical for animals in both propofol formulations studied.

Monitoring and Calculations:

Central venous pressure (CVP; mmHg) and pulmonary capillary wedge pressure (PCWP; mmHg) were determined using standard procedures. Mean arterial pressure (MAP; mmHg), MPAP and heart rate (HR; 1.min-1) were also continuously monitored. Cardiac output (CO; 1.min-1) was calculated by a standard thermodilution technique.

Body surface area (BSA; m2) was investigated using the Dubois equation:

BSA=body weight 0.425×body length (m) 0.725×0.007184.

The alveolo-arterial oxygen difference (A - a DO2) in arterial blood was calculated by using the equation:

(A−a DO2)=PAO2-PaO2

The arterial oxygen tension (PaO2) in arterial blood was measured, and the pulmonary endcapillary oxygen tension (PAO2) was calculated from the formula

PAO2=FiO2(PB−PH20)−PACO2 (FiO2+[1-FiO2/R])

where FiO2 is the inspired oxygen concentration, PB is ambient pressure, PH2O the water vapour pressure and R the respiratory quotient. The value of R used was 0.8. The alveolar carbon dioxide tension (PACO2) was assumed to equal PaCO2, the arterial carbon dioxide tension.

Haemodynamic parameters were calculated using the following equations. Volume related variables: Cardiac index (CI)=CO/BSA, stroke index (SI)=CI/HR. Flow related variables: Left ventricular stroke work index (LVSWI)=I×MAP, Right ventricular stroke work index (RSVWI)=SI×MPAP. The systemic vascular resistance index (SVRI) was calculated as: SVRI=(MAP−CVP)/CI×60, and pulmonary vascular resistance index (PVRI) as: PVRI=(MPAP−PCWP)/CI×60.

Laboratory Investigations:

1. Radioimmunoassay of 15-K-DH-PGF2α: Heparinized (unextracted) plasma samples were analysed for 15-K-DH-PGF2α as an index inflammatory response by radioimmunoassay (Basu S., Prostaglandins Leukot Essent Fatty Acids, 1998, 58, 347-352). The cross-reactivity of the antibody with PCF2α, 15-keto-PGF2α, PGE2 15-keto-13, 14-dihydro-PGE2, 8-iso-15-keto-13,14-dihydro-PGF2α, 11β-PGF2α, 9β-PGF2α, TXB2 and 8-iso-PGF2α was 0.02, 0.43,<0.001, 0.5, 1.7,<0.001,<0.001,<0.001, 0.01%, respectively. The detection limit was about 45 pmol/1.

2. Radioimmunassay of 8-iso-PGF2α: Heparinized (unextracted) plasma samples were analysed for 8-iso-PGF2α as an index of oxidative injury by radioimmunoassay (Basu S., Prostaglandins Leukot Essent Fatty Acids, 1998, 58, 319-325). The cross-reactivity of the 8-iso-PGF2α antibody with 15-keto-13,14-dihydro-8-iso-PGF2α, 8-iso-PGF2β, PGF2α, 15-keto-13,14-dihydro-PGF2α, TXB2, 11β-PGF2α, 9β-PGF2α and 8-iso-PGF3α respectively was 1.7, 9.8, 1.1, 0.01, 0.01, 0.1, 0.03, 1.8 and 0.6%. The detection limit of the assay was about 23 pmol/1.

3. Analysis of α-tocopherol and γ-tocopherol were analysed and correlated to triglycerides, and cholesterol, respectively. Arterial pH, base excess and blood gases were analysed by an ABL 300 (Radiometer, Copenhagen, Denmark) according to the manufacturer's recommendations.

Statistics

Differences in results between the two groups of pigs were calculated by a variance analysis test (ANOVA). The results were expressed as mean ±SD. A P value <0.05 was considered significant.

Results

Results are presented as follows:

FIGS. 1-6: plasma analysis for Experiment 1 (i.e. using original Diprivan). No plasma analysis has been performed for Experiment 2 (i.e. using modified Diprivan).

FIGS. 7-10: arterial pressure measurements for Experiments 1 and 2.

The following key has been used:

Original Diprivan (propofol without disodium edetate)=Prop or PPF

Modified Diprivan (propofol containing disodium edetate)=Prop+EDTA

Soya bean fat emulsion=Solvent=Solv

Endotoxin=Etx

Significance levels are indicated by the following key

* p<0.05

** p<0.01

*** p<0.001

In the group of 5 pigs dosed original Diprivan there were no deaths, in contrast to the control group of 5 pigs which received no propofol (i.e. soya bean fat emulsion alone) in which 2 pigs died.

In the group of 5 pigs dosed modified Diprivan there were no deaths, in contrast to the control group of 5 pigs which received no propofol (i.e. soya bean fat emulsion alone) in which 1 pig died.

The pigs that died did so because of multiple organ failure due to septic shock.

There were no differences in baseline values (weight, PaO2, cardiac performance, laboratory findings) between the pigs. Thus, animal physiological data (such as weight etc.) which were recorded to ensure maintenance of condition of the animals were essentially the same in all pigs whether using original or modified Diprivan, or a control.

### SUMMARY OF FIGURES

FIG. 1 shows Plasma concentrations of 15-keto-dihydro-prostaglandinF2a in endotoxemic pigs given original Diprivan or the corresponding volume of solvent.

The plasma 15-k-dh-PGF2α levels increased significantly within 30 minutes in the control group and remained high during the major part of the 6-hour long experiment. In the original Diprivan treated endotoxaemic pigs, plasma 15-k-dh-PGF2α levels increased to a lesser extent than the control group and returned to baseline levels soon.

Baseline values (=pre-endotoxaemic levels) at time zero reflect the ordinary conditions of anaesthetised pigs. Basal levels in non-anaesthetised pigs should be close to such levels.

FIG. 2 shows Plasma concentrations of 8-iso-prostaglandinF2a in endotoxemic pigs given original Diprivan or the corresponding volume of solvent

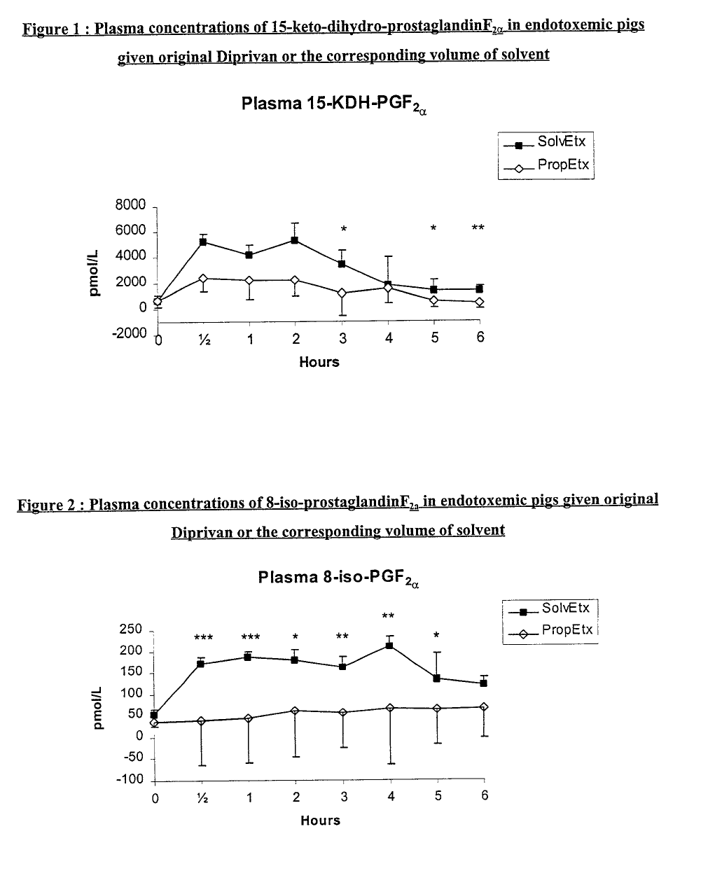

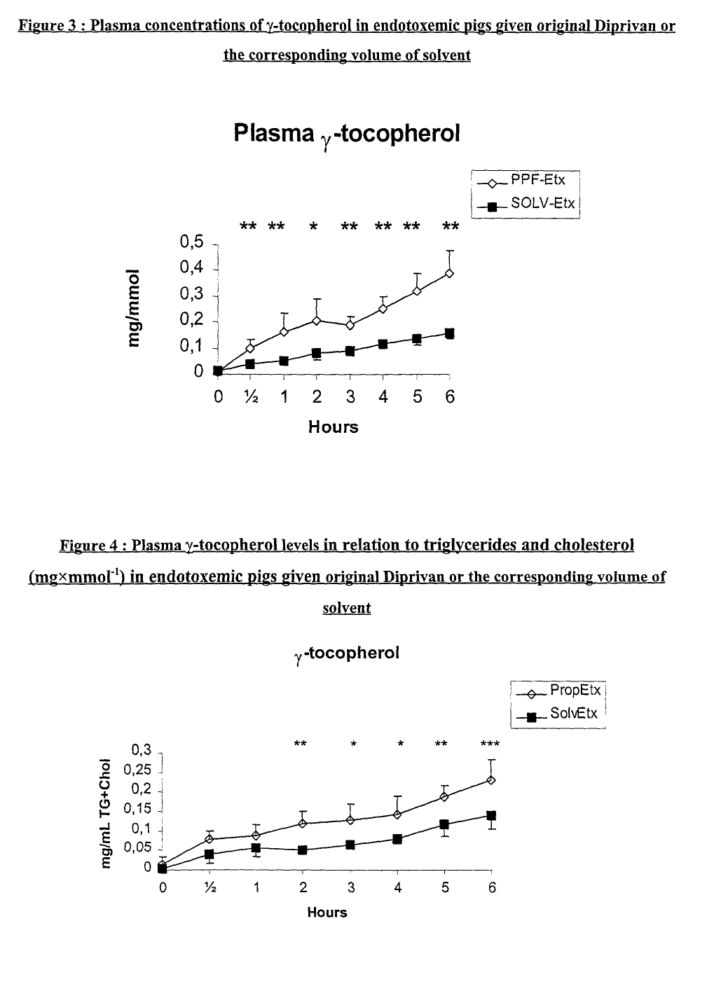

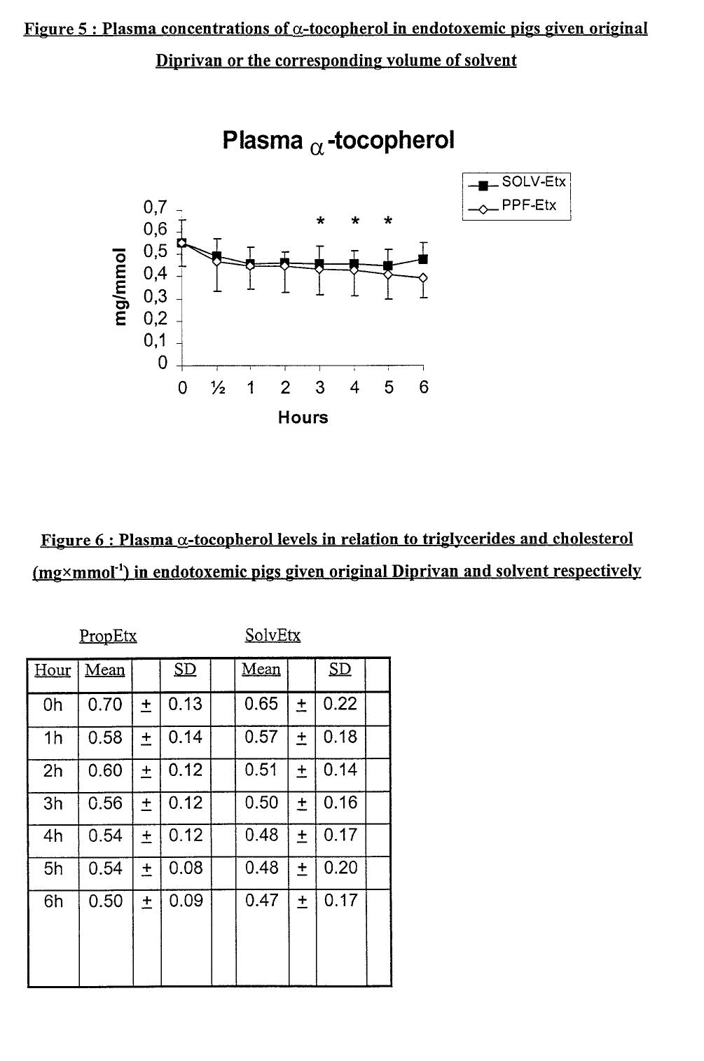

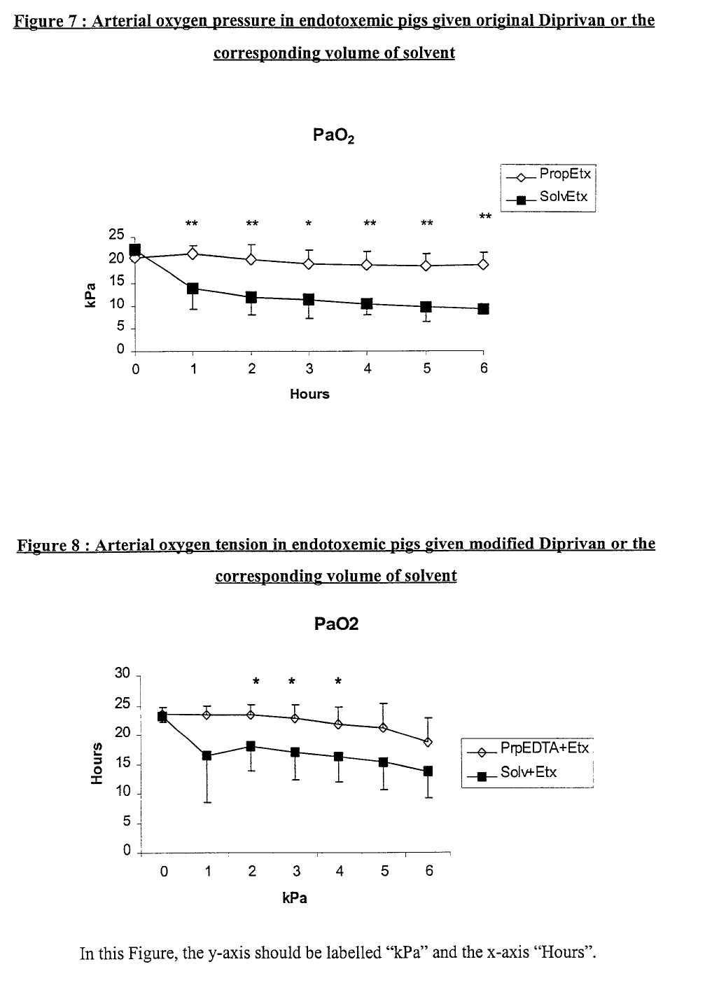

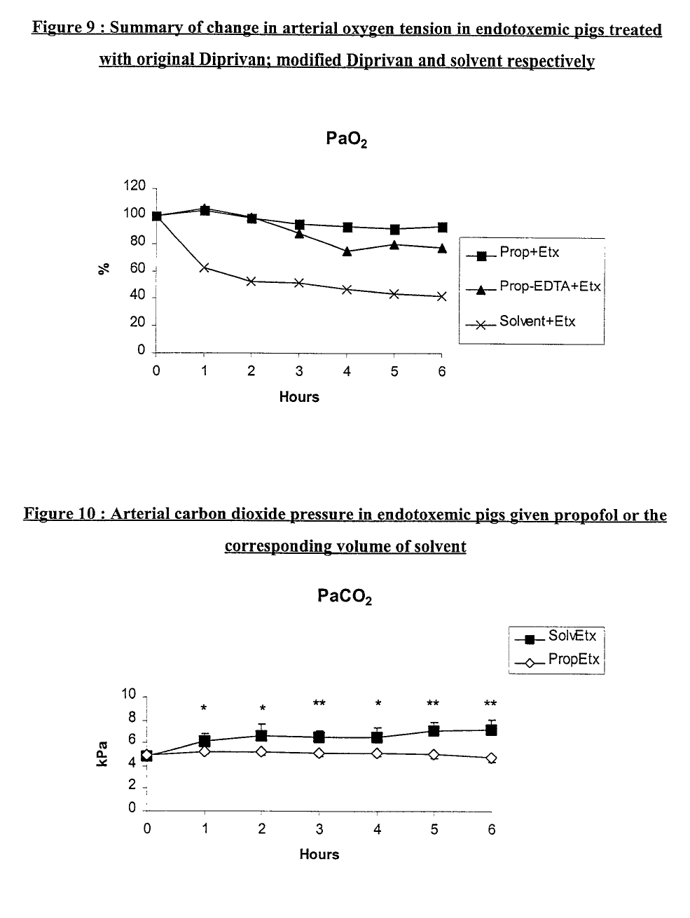

The plasma 8-iso-PGF2α levels increased significantly within 30 minutes in the control group and remained high during the major part of the 6-hour long experiment. No such increase of the 8-iso-PGF2α was seen in the original Diprivan treated endotoxaemic pigs. Thus, oxidative injury, as indicated by lower values of plasma 8-iso-PGF2α, was lower in the original Diprivan infused endotoxaemic pig as compared to controls given endotoxin+soya bean fat emulsion. Pulmonary vascular resistance index (PVRI) was lower in the original Diprivan+endotoxin infused group. Systemic and pulmonary haemodynamics were essentially the same in both groups.

Baseline values (=pre-endotoxaemic levels) at time zero reflect the ordinary conditions of anaesthetised pigs. Basal levels in non-anaesthetised pigs should be close to such levels.

FIG. 3 shows Plasma concentrations of γ-tocopherol in endotoxemic pigs given original Diprivan or the corresponding volume of solvent

FIG. 4 shows Plasma γ-tocopherol levels in relation to triglycerides and cholesterol (mg×mmol-1) in endotoxemic pigs given original Diprivan or the corresponding volume of solvent

FIG. 5 shows Plasma concentrations of α-tocopherol in endotoxemic pigs given original Diprivan or the corresponding volume of solvent

FIG. 6 : Plasma α-tocopherol levels in relation to triglycerides and cholesterol (mg×mmol-1) in endotoxemic pigs given original Diprivan and solvent respectively

| | |
|---|---|
| PropEtx | SolvEtx |

| Hour | Mean | | SD | Mean | | SD |
|---|---|---|---|---|---|---|
| 0 h | 0.70 | + | 0.13 | 0.65 | + | 0.22 |
| 1 h | 0.58 | + | 0.14 | 0.57 | + | 0.18 |
| 2 h | 0.60 | + | 0.12 | 0.51 | + | 0.14 |
| 3 h | 0.56 | + | 0.12 | 0.50 | + | 0.16 |
| 4 h | 0.54 | + | 0.12 | 0.48 | + | 0.17 |
| 5 h | 0.54 | + | 0.08 | 0.48 | + | 0.20 |
| 6 h | 0.50 | + | 0.09 | 0.47 | + | 0.17 |

Both the control soya bean emulsion and original Diprivan used contained α-tocopherol, β-tocopherol and γ-tocopherol, with the γ-tocopherol levels being much higher in each case (and with Vasolipid and Diprivan containing approximately equal levels of γ-tocopherol, although different batches may contain different levels). However, γ-tocopherol plasma levels increased significantly in the propofol treated group. It is possible that administration of Diprivan releases γ-tocopherol (possibly via a biotransformation from α- or β-forms to the γ-form of tocopherol). Also, since both Vasolipid and Diprivan contain γ-tocopherol, it may also be suggested that exogenously added γ-tocopherol is consumed to a higher rate in the control (=Vasolipid) group as compared to the Diprivan groups. Both Diprivan and γ-tocopherol act as scavengers which counteract free radical mediated injury. This type of injury can be evaluated as increased levels of 8-iso-PGF2α.

FIG. 7 shows Arterial oxygen pressure in endotoxemic pigs given original Diprivan or the corresponding volume of solvent

FIG. 8 shows Arterial oxygen tension in endotoxemic pigs given modified Diprivan or the corresponding volume of solvent

N.B. In this FIG. 8, the y-axis should be labelled “kPa” and the x-axis “Hours”.

FIG. 9 shows Summary of change in arterial oxygen tension in endotoxemic pigs treated with original Diprivan; modified Diprivan and solvent respectively

FIG. 10 shows Arterial carbon dioxide pressure in endotoxemic pigs given propofol or the corresponding volume of solvent

The best measure of the effect of propofol on septic shock is a measure of PaO2, and this is illustrated in FIGS. 7-10 above.

A (clinically) significant effect of Diprivan on PaO2 in septic shock is considered to be return towards (normalisation of) the pre-endotoxaemic (baseline) PaO2-levels. Any deterioration in PaO2 in the Diprivan-treated endotoxaemic is considered “insignificant”. This is in contrast to the deterioration in PaO2 seen in the control group.

PaO2 was higher, and PaCO2 was lower during the endotoxemic period in pigs given propofol instead of fat emulsion. Thus, propofol (whether in original or modified Diprivan) is effective in counteracting endotoxin induced deterioration of arterial oxygen tension (PaO2).

What is claimed is:

1. A sterile pharmaceutical composition for parenteral administration which comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal, for use as a medicament for managing septic shock.

2. A sterile pharmaceutical composition for parenteral administration according to claim 1, in which the sterile pharmaceutical composition comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination), for use as a medicament for managing septic shock.

3. The use of the compound 2,6-diisopropylphenol (propofol) for the manufacture of a medicament for managing septic shock.

4. The use of the compound 2,6-diisopropylphenol (propofol) for the manufacture of a medicament for counteracting endotoxin induced deterioration of arterial oxygen tension.

5. The use of a sterile pharmaceutical composition for parenteral administration which comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal, for the manufacture of a medicament for managing septic shock.

6. The use of a sterile pharmaceutical composition for parenteral administration which comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination) for the manufacture of a medicament for managing septic shock.

7. The use of a sterile pharmaceutical composition according to claim 5 or 6, for the manufacture of a medicament for counteracting endotoxin induced deterioration of arterial oxygen tension.

8. The use according to any one of claims 5 to 7, in which the sterile pharmaceutical composition is in the form of an oil-in-water emulsion which comprises:
(a) 1% by weight of propofol,
(b) 10% by weight of soy bean oil,
(c) 1.2% by weight of egg phosphatide,
(d) 2.25% by weight of glycerol,
(e) sodium hydroxide,
(f) water.

9. The use according to any one of claims 5 to 7, in which the sterile pharmaceutical composition is in the form of an oil-in-water emulsion which comprises:
(a) 2% by weight of propofol,
(b) 10% by weight of soy bean oil,
(c) 1.2% by weight of egg phosphatide,
(d) 2.25% by weight of glycerol,
(e) sodium hydroxide,
(f) water.

10. The use according to claim 8 or 9, in which the sterile pharmaceutical composition additionally contains 0.005% by weight of disodium edetate.

11. A method of managing septic shock which comprises administration of an effective amount of a sterile pharmaceutical composition for parenteral administration which composition comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal.

12. A method according to claim 11, in which the sterile pharmaceutical composition comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination).

13. A method of counteracting endotoxin induced deterioration of arterial oxygen tension which comprises administration of an effective amount of a sterile pharmaceutical composition for parenteral administration, which composition comprises the compound 2,6-diisopropylphenol (propofol) in association with a sterile pharmaceutically-acceptable diluent or carrier, the composition being suitable either directly or after dilution with a liquid diluent for parenteral administration to a warm-blooded animal.

14. A method according to claim 13, in which the sterile pharmaceutical composition comprises an oil-in-water emulsion in which propofol dissolved in a water-immiscible solvent, is emulsified with water and stabilised by means of a surfactant, and which optionally further comprises an amount of edetate sufficient to prevent significant growth of microorganisms for at least 24 hours (in the event of adventitious, extrinsic contamination).
